(19) United States
(12) Patent Application Publication
(10) Pub. No.: US 2008/0274066 A1
(43) Pub. Date: Nov. 6, 2008

(54) Compositions, Methods, Devices, and Kits for Maintaining or Enhancing Tooth Whitening

(75) Inventor: Robert Eric Montgomery, Monterey, MA (US)

Correspondence Address: DISCUS DENTAL, LLC, 8550 HIGUERA STREET, CULVER CITY, CA, 90232, US

(73) Assignee: Britesmile Professional, Inc.

(21) Appl. No.: 10/568,054

(22) PCT Filed: Jul. 28, 2004
(86) PCT No.: PCT/US04/24070

§ 371 Date: Feb. 12, 2007

Related U.S. Application Data
(63) Continuation of application No. 10/670,950, filed on Sep. 25, 2003.
(60) Provisional application No. 60/490,654, filed on Jul. 28, 2003.
(60) Provisional application No. 60/495,043, filed on Aug. 14, 2003.
(60) Provisional application No. 60/490,654, filed on Jul. 28, 2003.
(60) Provisional application No. 60/495,043, filed on Aug. 14, 2003.
(60) Provisional application No. 60/511,462, filed on Oct. 15, 2003.

(51) Int. Cl. A61K 8/22; A61C 17/00

(52) U.S. Cl. 424/53; 433/215; 433/89

(57) ABSTRACT

Compositions, methods, devices, and kits for maintaining or enhancing tooth whitening in a subject are disclosed. The methods include applying a first tooth whitening composition to the teeth of the subject for a predetermined period of time and applying a second tooth whitening composition to the teeth of the subject in predetermined intervals, wherein at least one of the first and second tooth whitening compositions are dispensed from a dental delivery device. The kits include a first tooth whitening composition and a second tooth whitening composition, each including an oxidizing agent. The oxidizing agent of the first tooth whitening composition is present in a higher amount than the second tooth whitening composition, which maintains or enhances the tooth whitening effect of the first tooth whitening composition.

### FIELD OF THE INVENTION

Compositions, methods, devices, and kits for maintaining or enhancing tooth whitening are disclosed.

### BACKGROUND OF THE INVENTION

White teeth have long been considered cosmetically desirable. Unfortunately, due to the presence of chromogenic (color-causing) substances in food, beverages, tobacco, and salivary fluid, in addition to internal sources such as blood, amalgam restoratives, and antibiotics such as tetracycline, teeth become almost invariably discolored in the absence of intervention. The tooth structures that are generally responsible for presenting a stained appearance are enamel, dentin, and the acquired pellicle.

Among the chemical strategies available for removing or destroying tooth stains, the most effective compositions contain an oxidizing agent, such as hydrogen peroxide, in order to attack the chromogen molecules in such a way as to render them colorless, water-soluble, or both. In one of the most popular approaches to whitening a patient's teeth, a dental professional will construct a custom-made tooth-bleaching tray for the patient from an impression made of the patient's dentition and prescribe the use of an oxidizing gel to be dispensed into the tooth-bleaching tray and worn intermittently over a period of time ranging from about 2 weeks to about 6 months, depending upon the severity of tooth staining. These oxidizing compositions, usually packaged in small plastic syringes, are dispensed directly by the patient, into the custom-made tooth-bleaching tray, held in place in the mouth for contact times of greater than about 60 minutes, and sometimes as long as 8 to 12 hours. The slow rate of bleaching is in large part the consequence of the very nature of formulations that are developed to maintain stability of the oxidizing composition.

Prolonged exposure of teeth to bleaching compositions, as practiced at present, has a number of adverse effects in addition to that of tooth sensitivity. These include: solubilization of calcium from the enamel layer at a pH less than 5.5 with associated demineralization; penetration of the intact enamel and dentin by the bleaching agents, so as to reach the pulp chamber of a vital tooth thereby risking damage to pulpal tissue; and dilution of the bleaching compositions with saliva resulting in leaching from the dental tray and subsequent ingestion.

To shorten exposure times, dental professionals began offering tooth whitening treatments utilizing oxidizing compositions (generally those with relatively high concentrations of oxidizers) which are applied directly to the tooth surface of a patient in a dental office setting. Theoretically, such tooth whitening strategies have the advantage of yielding faster results and better overall patient satisfaction. In-office procedures have been found to be quick and effective, but in some instances, patients have complained of a quick regression back to the original tooth shade prior to the in-office procedure. Maintenance products have been offered to help lengthen the tooth whitening effect; however, these products often require the use of dental trays. As described above, however, dental trays are cumbersome and patient compliance is oftentimes poor.

There is thus a need for improved compositions, methods, devices, and kits for whitening teeth that overcome the limitations of the prior art described above. In particular, there is a need for tooth whitening compositions and methods capable of whitening teeth quickly and safely, while providing a maintenance program with which a patient can easily comply. The compositions, methods, devices, and kits of the present invention described herein satisfy these and other needs.

### SUMMARY OF THE INVENTION

The present invention relates to compositions, methods, devices and kits for whitening teeth. The kits include a first tooth whitening composition that is applied to the teeth of a subject for a predetermined period of time and a second tooth whitening composition that is applied to the teeth of the subject in predetermined intervals, wherein at least one of the first and second tooth whitening compositions are dispensed in a dental delivery device.

Other compositions, kits, methods, devices, features, and advantages of the present invention will be or become apparent to one with skill in the art upon examination of the following drawings and detailed description. It is intended that all such additional compositions, kits, methods, devices, features, and advantages be included within this description, be within the scope of the present invention, and be protected by the accompanying claims.

### BRIEF DESCRIPTION OF THE DRAWINGS

FIG. 1 is a graph depicting the viscometric properties of the therapeutic dental gel composition of the present invention (BTG) in comparison with two prior art gels.

FIG. 2 depicts one embodiment of a delivery device of the present invention.

FIG. 3 depicts a felt tip pen that may be utilized as a device for administering the therapeutic dental composition of the present invention.

FIG. 4 depicts a brush pen that may be utilized as a device for administering the therapeutic dental composition of the present invention.

FIG. 5 is a graph depicting the viscosities of several gel products diluted with water.

FIG. 6 is a bar graph illustrating the shade change of Group A subjects utilizing a composition of the present invention.

FIG. 7 is a bar graph illustrating the shade change of Group B subjects utilizing a composition of the present invention.

FIG. 8 is a bar graph illustrating the sensitivity reported by patients utilizing a composition of the present invention.

FIG. 9 depicts a partial cross section of one embodiment of a delivery device of the present invention.

FIG. 10 depicts a partial cross section of one embodiment of a delivery device of the present invention.

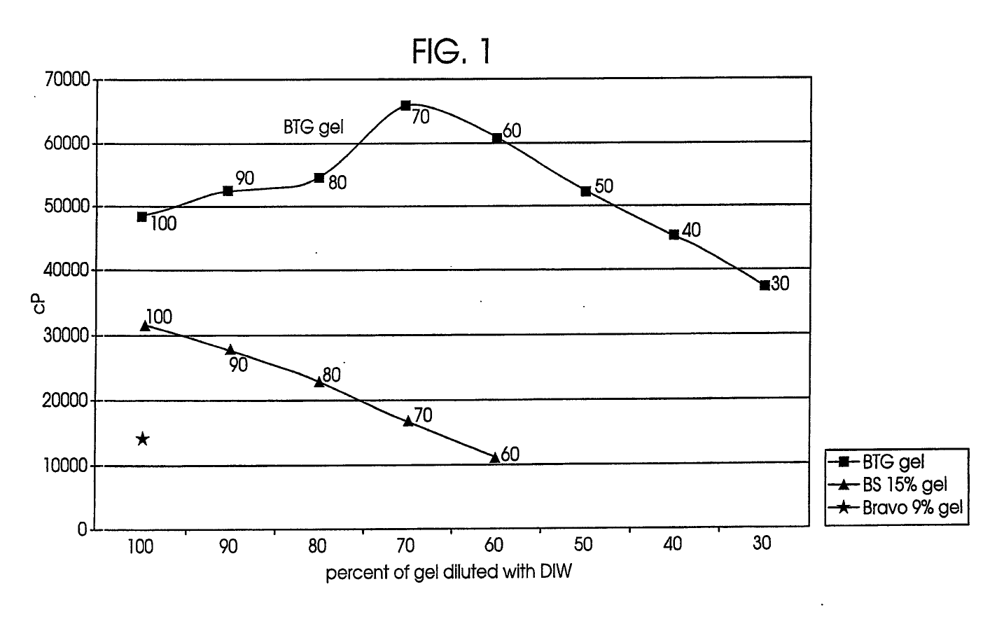

### DETAILED DESCRIPTION OF THE DRAWINGS

This section details the preferred embodiments of the subject invention. These embodiments are set forth to illustrate the invention, but are not to be construed as limiting. Since the present disclosure is directed to those skilled in the art field and is not a primer on the manufacture of tooth whitening compositions or their use or on devices for using such compositions, basic concepts and standard features known to those skilled in the art are not set forth in detail. Details for concepts such as choosing appropriate construction materials or ingredients, operating conditions or manufacturing techniques, etc. are known or readily determinable to those skilled in the art. Attention is directed to the appropriate texts and references known to those skilled in the art for details regarding these and other concepts which may be required in the practice of the invention; see, for example, Kirk-Othmer Encyclopedia of Chemical Technology, 4th Edition, Volumes 4 (1992), 13 (1995), 18 (1996), John Wiley & Sons, NY; Goldstein and Garber, Complete Dental Bleaching, Quintessence Publishing Co. 1995; and the aforementioned Journal of the American Dental Association, Vol. 128, Special Supplement, April 1997.

As used herein, the phrase “dental personnel” refers to dentists, other qualified dental professionals such as a registered dental hygienist or registered dental assistant, or any other employee of a dental practice that is authorized to perform tooth whitening services or provide instructions to dental patients.

The term “matrix” is defined herein as the gel, paste, or liquid in which an oxidizing agent is placed for administration to a subject.

The term “subject” referred to herein means mammals, including but not limited to humans and domestic animals.

The phrase “tooth surface” means a portion of a tooth which is directly responsible for the stained appearance of the tooth. The term tooth surface generally means a tooth's acquired pellicle, plaque, enamel, and combinations thereof.

Tooth whitening compositions, methods and kits, disclosed herein, provide a longer period of efficacious results than prior art methods and kits. In one aspect of the present invention, an initial high strength tooth whitening composition is applied onto the tooth surface of a subject and then subsequently a lower strength tooth whitening composition is applied onto the treated tooth surface. The first and second tooth whitening compositions each include an oxidizing agent that results in a whitening of the teeth. The first tooth whitening composition may include a higher concentration of the oxidizing agent than the second tooth whitening composition. Application of the first tooth whitening composition may provide an initial efficacious tooth whitening treatment that is maintained by subsequent application of the second tooth whitening composition. Alternatively, the second tooth whitening composition may contribute to an enhanced tooth whitening effect that is greater than application of the first tooth whitening composition alone.

In one aspect of the invention, the first and/or second tooth whitening compositions may be dispersed in a responsive gel which may be dispensed from a device. The tooth whitening composition can be held in the hand and used by a patient in need of tooth whitening, or by a separate individual, such as a dentist, to apply to the oral cavity of a patient. In the case of patient self-use, it is advantageous, but not required, for the patient to use the tooth whitening composition to apply the oxidizing agent to the teeth by using a mirror to guide placement and contact of the tooth whitening composition in the mouth.

The tooth whitening composition can be held directly by the patient or dentist, or alternatively the tooth whitening composition may be placed in a holder or other such device. In either case, the tooth whitening composition may be placed in direct contact with the tooth surface, or alternatively it may be first placed in or on a delivery device, such as a dental tray or strip, said delivery device then used to carry the tooth whitening composition into the oral cavity and thus into contact with the tooth surface. In another embodiment, the tooth whitening composition is applied directly onto the tooth surface and is immediately covered with a delivery device, such as a dental tray or plastic strip, in order to confine the composition to the area on the tooth surface or surfaces where it was applied. In yet another embodiment, prior to application to the oral cavity surface, the tooth whitening composition has a relatively low viscosity which permits easy dispensing from the delivery device. When applied to the oral cavity surface, the tooth whitening composition increases in viscosity to provide a more dilution-resistant gel when in contact with the tooth surface. The oxidizing agent will be released from the gel over a period of time.

In one embodiment, the first and/or second tooth whitening composition of the invention is comprised of a responsive gel carrier and at least one oxidizing agent dispersed throughout the carrier. The oxidizing agent may be dissolved in the responsive gel carrier or simply dispersed homogeneously in the carrier as an insoluble suspended solid particulate. The oxidizing agent may also be emulsified with the responsive gel carrier, creating separate and discrete carrier and oxidizing agent phases within the composition. The emulsion may be either an agent-in-carrier emulsion or a carrier-in-agent emulsion, analogous to an oil-in-water or a water-in-oil emulsion.

In one embodiment, the first and/or second tooth whitening composition includes: (1) a pharmaceutically acceptable, responsive gel carrier, (2) an oxidizing agent that is dissolved, dispersed or otherwise homogeneously distributed throughout the responsive gel carrier for the purpose of whitening a tooth surface; and (3) optionally, auxiliary ingredients such as flavorants, humectants, sweeteners, surface active agents, pH adjusting agents, stabilizing agents, secondary therapeutic agents, opacifying agents, colorants and other product modifying or enhancing components.

The use of a responsive gel carrier increases the viscosity of the tooth whitening composition when applied to a tooth surface, thereby forming a more viscous gel and increasing the oxidizing agent's contact time with the tooth surface. Once in contact with the tooth surface, the tooth whitening composition is then activated by the moisture in saliva by solubilizing, mobilizing or otherwise activating the oxidizing agent dispersed in the carrier. The oxidizing agent thus slowly migrates out of the viscous gel in the direction of the tooth surface, exerting a tooth whitening effect.

Longer contact times of the oxidizing agent with the tooth surface are achieved by the responsive gel carrier over less viscous or non-responsive compositions of the prior art. Lower concentrations of the oxidizing agent are thus possible than are conceivable with less viscous or non-responsive compositions, as much of the oxidizing agent in a less viscous composition quickly migrates away from the tooth surface after being dispensed to the oral cavity and/or solubilized in saliva.

Oxidizing Agent: The first and/or second tooth whitening compositions may include the same or a different oxidizing agent. Useful oxidizing agents that may be utilized in the first and/or second tooth whitening compositions of the present invention preferably include a peroxide, an alkali metal percarbonate, an alkali metal perborate, or a peroxyacid known in the art. Such oxidizing agents include, but are not limited to, hydrogen peroxide, carbamide peroxide, calcium peroxide, magnesium peroxide, zinc peroxide, sodium percarbonate, potassium percarbonate, potassium persulfate, sodium persulfate, ammonium persulfate, disodium monoperphosphate, dipotassium monoperphosphate, peroxyacids, magnesium monoperoxyphthalate, sodium perborate, chlorine dioxide, and sodium chlorite. Other oxidizing agents include materials that release hydrogen peroxide upon contact with water, such as an oxidoreductase enzyme and its corresponding substrate, for instance glucose oxidase and glucose. Ozone may also be used alone or in conjunction with one or more of the oxidizing agents listed herein. Often, it may be desirable to utilize a peroxyacid compound, such as peroxyacetic acid (for instance, when attempting to eliminate highly intractable tooth stains caused by tetracycline) in the tooth whitening composition. The peroxyacid may be included directly within the oxidizing composition. Alternatively, the peroxyacid may be formed by combining two or more separate phases (one of which contains a peroxyacid precursor, such as glyceryl triacetate and a second that contains one of the oxidizing agents listed above) prior to application to the tooth surface. Preferably, the peroxyacid is formed in situ, by contacting the tooth surface with a peroxyacid precursor prior to the application of an oxidizing agent; the peroxyacid is thus formed only on and within the stained tooth structure, where it is most beneficial to the tooth whitening process. Suitable peroxyacid precursors include, but are not limited to, glyceryl triacetate, acetylated amino acids, acetylsalicylic acid, and N,N,N′,N′-tetraacetyl ethylenediamine, vinyl acetate polymers and copolymers, acetylcholine, and other biologically acceptable acetylated compounds.

The oxidizing agent may be present in the first tooth whitening composition in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20.5%, 21.0%, 21.5%, 22.0%, 22.5%, 23.0%, 23.5%, 24.0%, 24.5%, 25%, 25.5%, 26.0%, 26.5%, 27.0%, 27.5%, 28.0%, 28.5%, 29.0%, 29.5%, 30.0%, 30.5%, 31.0%, 31.5%, 32.0%, 32.5%, 33.0%, 33.5%, 34.0%, 34.5%, 35.0%, 35.5%, 36.0%, 36.5%, 37.0%, 37.5%, 38.0%, 38.5%, 39.0%, 39.5%, 40.0%, 40.5%, 41.0%, 41.5%, 42.0%, 42.5%, 43.0%, 43.5%, 44.0%, 44.5%, 45.0%, 45.5%, 46.0%, 46.5%, 47.0%, 47.5%, 48.0%, 48.5%, 49.0%, 49.5%, 50% weight to weight of the first tooth whitening composition.

The oxidizing agent may be present in the second tooth whitening composition in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20.5%, 21.0%, 21.5%, 22.0%, 22.5%, 23.0%, 23.5%, 24.0%, 24.5%, 25%, 25.5%, 26.0%, 26.5%, 27.0%, 27.5%, 28.0%, 28.5%, 29.0%, 29.5%, 30.0%, 30.5%, 31.0%, 31.5%, 32.0%, 32.5%, 33.0%, 33.5%, 34.0%, 34.5%, 35.0%, 35.5%, 36.0%, 36.5%, 37.0%, 37.5%, 38.0%, 38.5%, 39.0%, 39.5%, 40.0%, 40.5%, 41.0%, 41.5%, 42.0%, 42.5%, 43.0%, 43.5%, 44.0%, 44.5%, 45.0%, 45.5%, 46.0%, 46.5%, 47.0%, 47.5%, 48.0%, 48.5%, 49.0%, 49.5%, 50% weight to weight of the second tooth whitening composition.

In one aspect of the present invention, the first tooth whitening composition includes a higher percentage of an oxidizing agent than the second tooth whitening composition. For example, the first tooth whitening composition may include a first oxidizing agent in an amount from about 10.0% to about 36.0% weight to weight of the first tooth whitening composition and the second tooth whitening composition may include a second oxidizing agent in an amount from about 1.0% to about 10.0% weight to weight of the second tooth whitening composition. In one embodiment, the first oxidizing agent is hydrogen peroxide in an amount of about 15.0% weight to weight of the first tooth whitening composition and the second oxidizing agent is hydrogen peroxide in an amount of about 5.0% weight to weight of the second tooth whitening composition.

The first oxidizing agent is dispersed throughout the first tooth whitening composition in a first matrix. Similarly, the second oxidizing agent is dispersed throughout the second tooth whitening composition in a second matrix. The first and second matrix include a carrier and, optionally, secondary therapeutic agents and/or auxiliary ingredients.

Carriers: The oxidizing agents of the first and second tooth whitening compositions are delivered to the tooth surface in a carrier. The carrier materials that can be employed in making the compositions of the present invention are any of those commonly used excipients in oral health and should be selected on the basis of compatibility with the oxidizing agent and the release profile properties of the desired delivery form.

Thickeners such as neutralized carboxypolymethylene and other polyacrylic acid polymers and copolymers, hydroxypropylcellulose and other cellulose ethers, salts of poly(methyl vinyl ether-co-maleic anhydride), polyvinyl pyrrolidone (PVP), poly(vinylpyrrolidone-co-vinyl acetate), silicon dioxide, fumed silica, stearic acid esters, and others are found to have utility in as carriers of the first and second tooth whitening compositions. Polymers utilized as thickeners may also serve as film-forming agents that provide for even distribution of the tooth whitening composition over the tooth surface. It is to be understood that additional useful thickeners will become apparent to those skilled in the art based upon the disclosure herein.

The level of thickener, when present, is highly dependent upon the type chosen, but in general is included in the composition at a concentration of from about 0.1% to about 20.0% or more by weight of the composition, and preferably at a concentration of from about 0.1% to about 5% by weight of the composition.

Water may also serve as a carrier in the first and/or second tooth whitening compositions. Water may be present in the first and/or second tooth whitening compositions in an amount of from about 60.0% to about 99.99% by weight of the composition. More particularly, water may comprise from about 70.0% to about 95.0% by weight of the first and/or second tooth whitening compositions.

Alternatively, the carrier may be a responsive gel carrier that contains any number of ingredients that alter the viscosity of a composition in response to the presence of moisture or in response to changes in temperature, pH, and/or ionic strength. The carrier of the present invention may include one or more ingredients that are sensitive to the presence of moisture or to changes in temperature, pH, or ionic strength. Examples of ingredients that are sensitive to the presence of moisture are complexes of high molecular weight acid functional polymers in combination with vinylpyrrolidone polymers (such as polyvinylpyrrolidone (PVP)) and copolymers. Surprisingly, it has been found that aqueous solutions of high concentrations of carboxypolymethylene, in the presence of PVP, do not achieve the high viscosities normally observed when adjusted to a pH range of between about 4.0 and 7.0. Upon dilution with water, however, these carboxypolymethylene/PVP complexes surprisingly demonstrate an increase in viscosity, rather than a decrease in viscosity as would be expected of most aqueous compositions. In particular, such carboxypolymethylene/PVP complexes achieve unexpectedly low viscosities in the presence of water-soluble salts, including but not limited to alkali metal salts such as sodium and potassium salt and/or ammonium salt. The increase in viscosity of this novel complex upon contact with moisture (for instance from saliva or residing as a film on a tooth or gum surface) has great utility in formulating the moisture-responsive dental carriers included in the tooth whitening compositions of the present invention.

Prior to exposure with water, the responsive gel carriers have low viscosity to permit easy dispensing of the first and/or second tooth whitening composition from a delivery device. The viscosity depressive effect of the carboxypolymethylene/PVP complex carrier is dependent upon the presence of water soluble salts. Without the presence of the water soluble salts, the complex carrier exhibits a high viscosity. Water soluble salts that may be utilized in maintaining a low viscosity in the carriers of the tooth whitening compositions of the present invention include but are not limited to sodium saccharin, sodium chloride, potassium chloride, and ammonium chloride may be utilized in the present invention as the source of water soluble salts.

A moisture sensitive polymer or polymer complex may be present in an amount of from about 0.01 to about 20% (w/w) of the first and/or second tooth whitening composition, more preferably from about 0.01% to about 10% (w/w) of the tooth whitening composition. More particularly, the concentration of moisture sensitive polymer or polymer complex in the tooth whitening composition may be about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20.5%, 21.0%, 21.5%, 22.0%, 22.5%, 23.0%, 23.5%, 24.0%, 24.5%, 25%, 25.5%, 26.0%, 26.5%, 27.0%, 27.5%, 28.0%, 28.5%, 29.0%, 29.5%, 30.0% weight to weight of the tooth whitening composition.

As used herein, pH sensitive polymers mean any polymer that gels in response to an increase in pH. Examples of water-soluble ingredients sensitive to pH and ionic strength include, but are not limited to, carboxypolymethylene (Carbopol®, Noveon), hydrolyzed or unhydrolyzed PVP/maleic acid anhydride copolymer (Gantrez, ISP), polycarboxylates, gellan gum (Gelrite, CP Kelco), poly(methyl methacrylate-co-methacrylic acid) (such as Eudragit, Rohm Pharma), hydroxypropyl methylcellulose phthalate, and cellulose acetate phthalate. Suitable polycarboxylates include but are not limited to polymers and copolymers of acrylic acid, methacrylic acid, maleic acid (or maleic anhydride), fumaric acid, itaconic acid, aconitic acid, mesaconic acid, citraconic acid and methylenemalonic acid, mellitic acid, succinic acid, oxydisuccinic acid, polymaleic acid, benzene 1,3,5-tricarboxylic acid, carboxymethyloxysuccinic acid, and soluble salts thereof.

As used herein, temperature sensitive polymer shall mean any polymer that gels in response to increases in temperature above about 30 degrees Celsius. Temperature sensitive ingredients may include but are not limited to methylcellulose, hydroxypropyl methylcellulose, ethyl(hydroxyethyl)cellulose (in the presence of ionic surfactants), and polyoxyethylene-polyoxypropylene block copolymers (such as Pluronic F-127 and F-108, BASF).

The pH or ion sensitive ingredient may be present in an amount of from about 0.01 to about 20% of the first and/or second tooth whitening composition, more preferably from about 0.01% to about 10% of the tooth whitening composition. More particularly, the concentration of pH or ion sensitive ingredient in the tooth whitening composition may be about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20.5%, 21.0%, 21.5%, 22.0%, 22.5%, 23.0%, 23.5%, 24.0%, 24.5%, 25%, 25.5%, 26.0%, 26.5%, 27.0%, 27.5%, 28.0%, 28.5%, 29.0%, 29.5%, 30.0% weight to weight of the tooth whitening composition.

The temperature sensitive ingredient may be present in an amount of from about 0.01% to about 20% of the first and/or second tooth whitening composition, more preferably from about 0.01% to about 10.0% of the tooth whitening composition. More particularly, the concentration of temperature sensitive ingredient in the tooth whitening composition may be about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20.5%, 21.0%, 21.5%, 22.0%, 22.5%, 23.0%, 23.5%, 24.0%, 24.5%, 25%, 25.5%, 26.0%, 26.5%, 27.0%, 27.5%, 28.0%, 28.5%, 29.0%, 29.5%, 30.0% weight to weight of the tooth whitening composition.

Additionally, the responsive gel carrier may include water in an amount of from about 1.0% to about 99.9% of the tooth whitening composition, more preferably from about 10.0% to about 98.7% of the tooth whitening composition. The responsive gel carrier may further include a polyol that assists in water retention and/or modifying the gelling temperature of the tooth whitening composition. Examples of polyols include but are not limited to glycerin, propylene glycol, polyethylene glycol, mannitol, sorbitol, maltitol, xylitol, lactitol and others. The polyol may be present in the tooth whitening composition in an amount from about 1.0% to about 50.0% (w/w).

The concentration of responsive gel carrier in the first and/or second tooth whitening composition may be about 10%, 15%, 20%, 25%, 30%, 35%, 40%, 45%, 50%, 55%, 60%, 65%, 70%, 75%, 80%, 85%, 90%, 95%, 96%, 97%, 98%, 99% weight to weight of the tooth whitening composition.

Secondary therapeutic agents: Secondary therapeutic agents contemplated to be included in the tooth whitening compositions of the present invention include antimicrobial agents, anti-inflammatory agents, tooth desensitizers, anticaries agents, tartar control agents, tooth and gum surface protectants, tooth stain prevention agents and agents effective against dental plaque, halitosis, gingivitis, periodontal disease, oral ulcers and other diseases, afflictions or symptoms of the oral cavity.

Suitable antimicrobial agents known or anticipated to have utility in the inventive compositions include compounds with inhibitory activity against microorganisms found in the oral cavity. Compounds such as triclosan, chlorhexidine salts (such as chlorhexidine digluconate), cetylpyridinium chloride and domiphen bromide are suitable antimicrobial agents useful in the present inventive compositions.

Suitable anticaries agents include but are not limited to a source of fluoride ion. Fluoride sources include sodium fluoride, potassium fluoride, calcium fluoride, amine fluorides, stannous fluoride, stannous monofluorophosphate and sodium monofluorophosphate. These sources should release anywhere from about 25 to about 3500 ppm of fluoride ion. The anti-caries agent may be present in an amount from about 0.05% to about 3.0%, preferably about 0.2% to about 1.0% by weight of the tooth whitening composition.

Suitable tartar control agents include but are not limited to zinc salts (e.g., zinc citrate trihydrate) and agents containing multiple phosphate moieties (e.g., sodium tripolyphosphate). Inorganic polyphosphate tartar control agents may include any of the pyrophosphates such as disodium pyrophosphate, dipotassium pyrophosphate, tetrapotassium pyrophosphate, tetrasodium pyrophosphate and mixtures thereof, as well as higher polyphosphates such as sodium tripolyphophate, sodium hexametaphosphate and mixtures thereof. Organic phosphorous compounds that may serve as tartar control agents include polyphosphonates such as disodium ethane-1-hydroxy-1,1-diphosphonate (EHDP), methanediphosphonic acid, and 2-phosphonobutane-1,2,4-tricarboxylic acid. Amounts of the polyphosphate may range from about 0.5% to about 20.0%, preferably from about 1.0% to about 8.0%, optimally from about 1.2% to about 4.5% by weight of the tooth whitening compositions of the present invention. As an alternative to phosphates, zinc salts may be utilized as anti-tartar agents. Most preferred is zinc citrate trihydrate. Amounts of the zinc salt may range from about 0.5% to about 20%, preferably from about 1.0 to about 8.0%, optimally from about 2.0% to about 6.0% by weight of the tooth whitening composition.

The concentration of the secondary therapeutic agents in the tooth whitening composition may be about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20.5%, 21.0%, 21.5%, 22.0%, 22.5%, 23.0%, 23.5%, 24.0%, 24.5%, 25%, 25.5%, 26.0%, 26.5%, 27.0%, 27.5%, 28.0%, 28.5%, 29.0%, 29.5%, 30.0%, 30.5%, 31.0%, 31.5%, 32.0%, 32.5%, 33.0%, 33.5%, 34.0%, 34.5%, 35.0%, 35.5%, 36.0%, 36.5%, 37.0%, 37.5%, 38.0%, 38.5%, 39.0%, 39.5%, 40.0%, 40.5%, 41.0%, 41.5%, 42.0%, 42.5%, 43.0%, 43.5%, 44.0%, 44.5%, 45.0%, 45.5%, 46.0%, 46.5%, 47.0%, 47.5%, 48.0%, 48.5%, 49.0%, 49.5%, 50% weight to weight of the tooth whitening composition.

Auxiliary Ingredients: Auxiliary ingredients contemplated to be included in the tooth whitening compositions of the present invention include flavorants, humectants, sweeteners, surface active agents, pH adjusting agents, stabilizing agents, opacifying agents, colorants and other product modifying or enhancing components.

Suitable flavorants include but are not limited to oils derived from plants and fruits such as citrus oils, fruit essences, mint, peppermint oil, spearmint oil, capsaicin, clove oil, oil of wintergreen, anise, sassafras, sage, eucalyptus, marjoram, cinnamon, lemon, orange, banana, cherry, apple, pineapple, grape, strawberry, blueberry, tutti frutti, methyl salicylate, Hagelin flavoring #640047, Hagelin flavoring #640057, Hagelin flavoring #671009, Hagelin flavoring #671010, and the like. Those skilled in the art will recognize that natural and artificial flavoring agents may be used independently or combined in any sensorially acceptable blend.

Suitable humectants include but are not limited to glycerin, sorbitol, xylitol, mannitol, lactitol, maltitol, and other sugar alcohols, polyethylene glycol, propylene glycol, and other edible polyhydric alcohols and mixtures thereof.

Suitable sweeteners include but are not limited to sucrose, lactose, dextrose, maltose, dextrin, dried inverted sugar, fructose, levulose, galactose, corn syrup and their solids, sorbitol, mannitol, xylitol, hydrogenated starch hydrolysates, maltitol, sucralose, aspartame, salts of acesulfame, alitame, saccharin and its salts, cyclamic acid and its salts, glycyrrhizin, dihydrochalcones, thaumatin, monellin, stevia extract and the like.

Suitable surface active agents include but are not limited to sodium lauryl sulfate, condensates of sorbitan mono-oleate with from about 20 to 60 moles of ethylene oxide (e.g., “Tweens” a trademark of ICI United States, Inc.), condensates of ethylene oxide with propylene oxide and condensates of propylene glycol (“Pluronics” a trademark of BASF-Wyandotte Corp.).

Suitable pH adjusting agents include but are not limited to sodium hydroxide, potassium hydroxide, ammonium hydroxide, sodium carbonate, potassium carbonate, sodium phosphate di- and tri-basic, potassium phosphate di- and tri-basic, sodium tripolyphosphate, tris(hydroxymethyl)aminomethane (“TRIS”), triethanolamine, and polyethylenimine.

The first and/or second tooth whitening composition may also contain a stabilizing agent. Suitable stabilizing agents include but are not limited to 1-hydroxyethylidene-1,1-diphosphonic acid (Dequest 2010), sodium stannate trihydrate, potassium stannate triphydrate, sodium acid pyrophosphate, ethylenediamine tetraacetic acid (EDTA), diethylenetriamine pentaacetic acid (DETPA), nitrilotriacetic acid (NTA), ethylenediamine tetra(methylenephosphonic acid), diethylenetriamine penta(methylenephosphonic acid), sorbitol, xylitol, mannitol, maltitol, lactitol, alkali metal pyrophosphates and alkali metal polyphosphates. In certain formulations, a single component may act either as a calcium chelating agent or as a stabilizing agent or may serve both functions. A calcium chelating agent prevents precipitation of calcium ions, especially at tooth surface pH levels greater than about 5.5. Examples of calcium chelating agents include any of the calcium chelating agents known in the art and include 1-hydroxyethylidene-1,1-diphosphonic acid, ethylenediamine tetra(methylenephosphonic acid), and diethylenetriamine penta(methylenephosphonic acid), NTA, DEPTA, EDTA and its salts, citric acid and its salts, gluconic acid and its salts, alkali metal pyrophosphates and alkali metal polyphosphates. Without being bound to any particular theory, it is proposed that calcium precipitation in the form of calcium phosphates arise in the intercrystalline interstices of the tooth at elevated pHs and this gives rise to a blockage of movement of the peroxide into the tooth with a resulting negative effect on tooth bleaching. Calcium chelating agents may prevent this precipitation of calcium ions with the associated observed improvement of tooth-bleaching effect.

Suitable opacifying agents include but are not limited to titanium dioxide and zinc oxide.

Suitable colorants include but are not limited to FD&C-type dyes and lakes, fruit and vegetable extracts, titanium dioxide, and the like, alone or in combination.

Additional carriers, therapeutic agents and auxiliary ingredients useful in the invention are listed in Remington's, The Science and Practice of Pharmacy (2000); Lieberman et al., Pharmaceutical Dosage Forms (2d ed. 1989); Merck Index (13th Ed.).

Suitable compositions that may also be utilized as the first and/or second tooth whitening compositions of the present invention are also disclosed in U.S. Pat. Nos. 5,922,307; 6,162,055; 6,221,341; and 6,479,037; and U.S. application Ser. No. 10/434,597, filed May 9, 2003; U.S. application Ser. No. TBA, filed Sep. 25, 2003, entitled “Therapeutic Responsive Dental Gel Composition”; and U.S. Provisional Application No. 60/472,427, filed May 16, 2003.

Each of the first and second tooth whitening compositions may be administered from a single component or multi-component device such as a syringe, tube, or vessel or from a dental delivery device such as a pen, pencil, or liquid stick having an applicator, such as a felt tip, brush, roller ball, or non-woven pad. Each of the first and second tooth whitening compositions may be dispensed from a delivery device into a dental tray or strip or directly onto the tooth surface.

In a two-component device, the mixing of the two components from a single tooth whitening composition can be readily achieved using a multi-component tube containing a baffle, otherwise known in the art as a static mixer such that on squeezing the tube, material from each of the compartments is forced through the static mixer and are mixed together before emerging from a single exit in the tube, as is disclosed in U.S. Pat. No. 6,536,628.

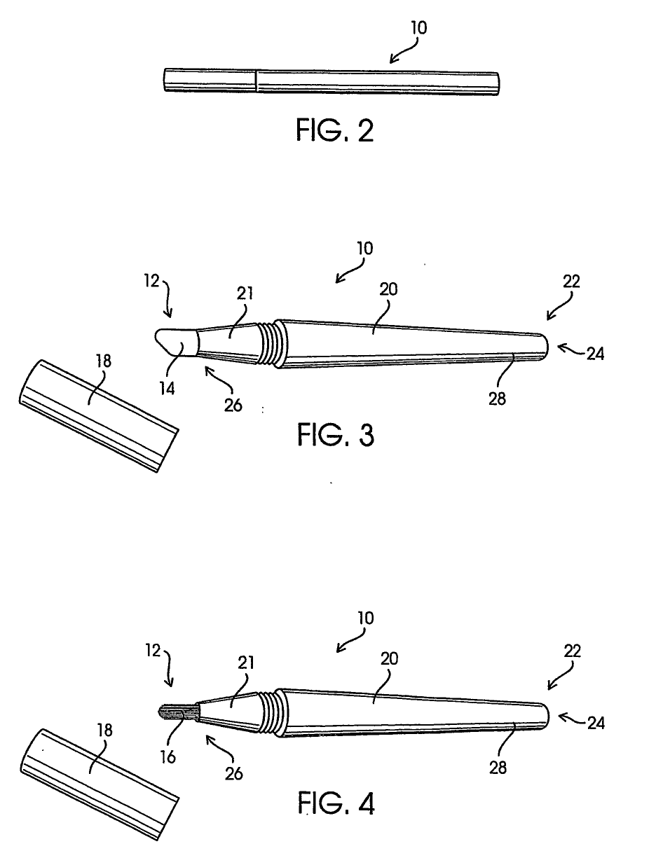

The first and/or second tooth whitening compositions are preferably disposed in a delivery device 10 (e.g., FIGS. 2-4, 9, and 10), such as a dispensing tube, pencil, pen or liquid stick having an applicator 12, such as a felt tip 14 (FIG. 3), brush 16 (FIG. 4), roller ball, or non-woven pad. In one embodiment, the delivery device 10 includes more than one applicator 12 that may be removably engaged with the device 10. In an embodiment wherein the device 10 is a pen or a pencil, the applicator 12 may be retractable and/or housed in a cap 18. The tooth whitening compositions of the present invention may be housed directly within a reservoir 20 in the device 10 or may be supplied in a removable cartridge (not shown) within the reservoir 20 that may be replaced or refilled. The delivery device 10 may dispense the tooth whitening composition through a transfer channel 21 through capillary action, such as in a flow through pen, or through an actuator 22, such as mechanical piston with a click mechanism, twist button and ratchet mechanism, or push button mechanism, or through a vacuum method of ejection, or through other such mechanical means for transferring the composition from the device to an oral cavity surface in need of treatment. The actuator 22 may be present on first end 24 of the device 10 and the applicator on a second end 26 of the device 10 or the actuator 22 may be present on a side wall 28 of the device. In one embodiment, the delivery device 10 includes a felt tip 14 or brush 16 applicator 12 wherein the inventive composition is dispensed to the applicator 12 through actuation of the actuator 22, such as by a clicking or twisting mechanism. Kotobuke Pencil, Japan, is one manufacturer of such types of delivery devices 10 (see, e.g., U.S. Pat. No. 6,176,632).

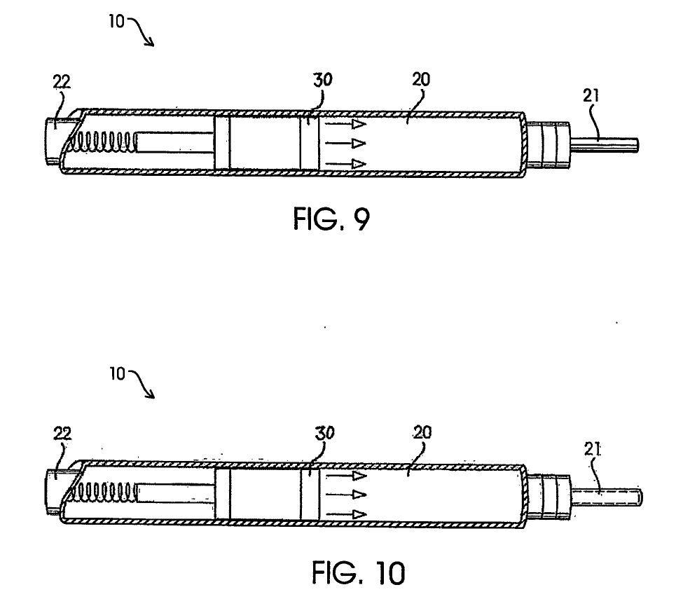

Preferably, the device 10 is free of metal components (e.g., FIG. 10), more preferably made of plastic components or metal components coated with plastic. In one embodiment, the device is made from fluoropolymers, polypropylene, polyethylene, or other such polymers that are compatible with the ingredients of the compositions of the present invention. In a preferred embodiment, all of the device components that are in contact with the tooth whitening composition, i.e., the plunger 30, the reservoir 20, the transfer channel 21 and the applicator 12, are all constructed out of plastic components (see FIG. 10), or metal components (see FIG. 9) that have been coated with plastic on those surfaces in contact with the tooth whitening composition, in order to improve the compatibility of the device 10 components that are in intimate contact with said peroxide-containing tooth whitening composition. (see FIGS. 9, 10).

Upon applying external pressure to the actuator 22 to expel the composition from the reservoir 20, the tooth whitening composition responds to shear forces introduced by the external pressure, and is temporarily reduced in viscosity to allow for ease of movement of the composition from the reservoir 20 through the transfer channel 21 to the applicator 12. Once the composition is positioned on the applicator 12, the user applies the composition to the teeth or gum surfaces, using the applicator 12 to apply and distribute the composition on the teeth and/or gums. Optionally, a set of instructions may be provided to the user in order that a particular application method or protocol be employed to apply the composition from the device 10 onto the teeth and/or gums in order to optimize the performance of the composition. With a twist mechanism, the user twists the actuator 22 on the first end 24 of the delivery device 10 and the tooth whitening composition travels from the reservoir 20 through the transfer channel 21 to the applicator 12 at the other end. With the push button actuator 22, the tooth whitening composition is delivered to the oral cavity surface with the push of a button actuator 22 on the first end 24 or side wall 28, which transfers the composition from the reservoir 20 through the transfer channel 21 to the applicator 12.

The delivery devices 10 of the present invention may deliver a dose of the tooth whitening composition upon each application to an oral cavity surface, for example, with each click or twist of the actuator mechanism 22. The dose includes from about 0.01 ml to about 3.0 ml of the composition, preferably from about 0.1 ml to about 1.0 ml, more preferably from 0.1 ml to about 0.5 ml, and most preferably from about 0.2 ml to about 0.3 ml of the composition. In one embodiment, the amount of dose dispensed from the device 10 may be adjusted by the user.

The tooth whitening compositions can be dispensed from any suitable delivery device 10 as described above. For example, the tooth whitening compositions may be dispensed as a liquid or thin gel from a push button or twist actuated pen with an advancing piston mechanism that expels a predetermined amount of liquid or gel through an orifice. The pen delivery device 10 just described may also optionally comprise a set of bristles, advantageously positioned near or around the orifice through which the therapeutic dental liquid or gel is expelled. Expelling the therapeutic liquid or gel through the orifice and onto said bristles, the user may apply the tooth whitening composition directly onto the teeth, thereby forming a thickened gel upon application. Alternatively, the tooth whitening compositions may be brushed onto an oral cavity surface, using a brush (FIG. 4) or felt tip (FIG. 3) that is replenished with the tooth whitening composition by returning it to a reservoir containing said composition or by clicking or twisting a dispensing portion of the reservoir.

The present invention further relates to a method of whitening teeth in a subject comprising applying a first tooth whitening composition to the teeth of the subject for an initial predetermined period of time and thereafter applying a second tooth whitening composition to the teeth of the subject for predetermined intervals of time. The first composition provides an initial tooth whitening effect that is maintained or enhanced by subsequent application of the second tooth whitening composition. The first tooth whitening composition is applied as the initial treatment for whitening the subject's teeth. The first tooth whitening composition may be applied to the subject's tooth surface from about thirty seconds to about two hours, preferably from about fifteen minutes to about forty-five minutes, more preferably from about twenty-five minutes to about thirty-five minutes, and most preferably for about 30 minutes. The first tooth whitening composition may be applied once to the subject's tooth surface or may be applied in intervals. For example, the first tooth whitening composition may be applied to the subject's tooth surface for three 20 minute intervals. In one embodiment, the first tooth whitening composition is applied to the subject's tooth surface by dental personnel in a dental office setting. In another embodiment, the subject applies the first tooth whitening composition to the subject's own tooth surface.

The second tooth whitening composition may then be used to maintain or enhance the tooth whitening effect of the first tooth whitening composition. Dental personnel may apply the second tooth whitening composition to the subject's tooth surface or may instruct the subject to apply the second tooth whitening composition after the initial treatment with the first tooth whitening composition. Application of the second tooth whitening composition may be performed, for instance, only once, or alternatively may be performed on a regularly scheduled basis, for instance once a day for fourteen days. Further, application of the composition may occur more than once a day for an extended period of time. For example, the second tooth whitening composition may be administered to a subject one to six times per day, for a period of time ranging from 1 to 180 days. In one embodiment, the composition is administered twice a day for 14 days. In another embodiment, the composition is administered once a day for 30 days. It is also contemplated that the inventive compositions may be used on a daily basis.

The methods of the present invention may require that dental personnel apply the first and/or second tooth whitening compositions to the subject's tooth surface. Alternatively, the dental personnel or instruction manual instructs the subject to apply the first and/or second tooth whitening compositions to the subject's tooth surface. In one embodiment, the dental personnel applies the first tooth whitening composition to the subject's tooth surface in a dental office. Subsequently, the dental personnel instructs the subject to apply the second tooth whitening composition to the subject's tooth surface outside of the dental office. When the first and/or second tooth whitening compositions are applied in the dental office, the dental personnel may place a cheek retractor and a bite block in the subject's mouth prior to application of the composition. Further, the dental personnel may apply barrier material to the oral cavity to protect the gingival margins from the oxidizing agent, particularly when using concentrations of oxidizing agent higher than about 10.0% weight to weight of the composition.

The methods of the present invention may further require that the subject brushes the teeth with pre-whitening toothpaste prior to application of the first tooth whitening composition.

One embodiment of the present invention includes each of the following steps:

(1) A barrier material to protect the gums from the oxidizing agent (supplied by BriteSmile, Inc., Walnut Creek, Calif.) is first applied to the upper first and second premolar gingival area starting at the gum line and tooth junction (actually contacting the enamel) and then cured for three seconds. The barrier material should be thick enough so that no pink gingival tissue is exposed. For every inch of isolation coverage, a standard curing light may be used for no more than three seconds per any given spot to solidify the barrier material. The application of the barrier material is continued over the entire upper arch and then repeated for the lower arch.
(2) The first tooth whitening composition is applied to the teeth 1 to 2 mm thick and any excess saliva is suctioned if necessary. After about 30 minutes, the first tooth whitening composition is suctioned from the patient's teeth.
(3) Once the procedure is finished, excess materials are removed from the patient, for example, cotton rolls, isolation material, optic positioner, excess barrier material, and cheek retractors. The teeth are then flushed thoroughly with water.
(4) If the patient experienced any discomfort during the treatment, or in the case of a young adult client, a neutral sodium fluoride treatment utilizing a white foam or clear neutral sodium fluoride may be administered.
(5) The patient is given the second tooth whitening composition and instructed to apply the second tooth whitening composition to the tooth surface twice per day for fourteen days.

The first and second tooth whitening compositions described above may be included in a two-component tooth whitening system or a kit for whitening teeth. In one embodiment, the kit includes a first tooth whitening composition comprising about 15.0% hydrogen peroxide weight to weight of the composition and a second tooth whitening composition comprising about 5.0% hydrogen peroxide weight to weight of the composition and a set of instructions. The set of instructions directs the dental personnel to apply the first tooth whitening composition to the subject's tooth surface for about 30 minutes. Thereafter, the instructions provide the dental personnel to instruct the subject to apply the second tooth whitening composition to the tooth surface twice per day for fourteen days. Alternatively, the instructions may directly instruct the subject on the application procedure without requiring dental personnel intervention. The first and second tooth whitening compositions of the kit may be supplied in a single component or multi-component device such as a syringe, tube, or vessel or in a dental delivery device as described above.

It is believed that one skilled in the art, based on the description herein, can utilize the present invention to its fullest extent. The following specific examples are therefore to be construed as merely illustrative, and not limitative of the remainder of the disclosure in any way whatsoever.

### EXAMPLES

### Example 1

The following first tooth whitening composition was prepared, which contained approximately 15% by weight hydrogen peroxide and 1.0% by weight of the photosensitizer precursor 1-hydroxyethylidene-1,1-diphosphonic acid (Dequest 2010, Monsanto Corp., St. Louis, Mo.). Highly purified water (18.2 megaohm, filtered through a 0.2 micron filter) was utilized in order to maintain good stability of the composition during storage. The composition was thickened with a carboxypolymethylene polymer (Carbopol 974P, B. F. Goodrich Co., Cleveland, Ohio) to the consistency of a light, non-runny gel. Glycerin was added in a small percentage as a humectant and stabilizer (as a free radical scavenger), and the Carbopol 947P was neutralized to a pH of 5.5 with ammonium hydroxide, resulting in the formation of a transparent and thixotropic gel.

| Ingredient | Percentage |
|---|---|
| Distilled water | 49.400 |
| 1-hydroxyethylidene-1,1-diphosphonic acid | 1.000 |
| Glycerin 99.7% | 5.000 |
| Hydrogen peroxide 35% | 42.900 |
| Carbopol 974P | 1.700 |
| Ammonium hydroxide 29% | to pH 5.5 |
| TOTAL | 100.000 |

The above composition was prepared in a plastic mixing chamber by combining, under agitation with a Teflon-coated mixing paddle until a clear solution was obtained, the distilled water, the 1-hydroxyethylidene-1,1-diphosphonic acid, and the glycerin. The Carbopol 974P was then sifted slowly into the vortex created by the mixing paddle and allowed to mix until a homogeneous slurry of the polymer was obtained. Finally, the ammonium hydroxide was added in a constant, drop-wise fashion over a period of about 5 minutes until thickening and clarification of the slurry occurred. A pH probe was inserted periodically and the ammonium hydroxide addition proceeded until a pH of exactly 5.5 was obtained. The resulting gel contained 15% by weight hydrogen peroxide, and was highly transparent and thixotropic (non-slumping) in character.

### Example 2

The following first tooth whitening composition was prepared according to the procedure of Example 1.

| Ingredient | Percent (w/w) |
|---|---|
| Deionized water | 77.2868 |
| Hydrogen peroxide | 15.0000 |
| Glycerin | 5.0000 |
| Carbopol 974P-NF | 1.7900 |
| 1-hydroxyethylidene-1,1-diphosphonic acid | 0.3000 |
| Potassium stannate trihydrate | 0.0200 |
| Ammonium hydroxide | 0.6032 |
| | 100.0000 |

### Example 3

Extracted human teeth (HE) that were non-carious and free of amalgam or resin-based restorative materials were utilized to study the ability of the composition of Example 1 to eliminate the strains from human enamel and dentin. The teeth were coated with a 1-2 mm thick film of the first tooth whitening composition of Example 1. The resulting change in tooth color (Δ Shades) was recorded as the number of VITA® (shade difference between the original baseline VITA® shade value and the final VITA® shade value.

TABLE 1

| | | Exposure | Shade | Shade | |
|---|---|---|---|---|---|
| Tooth # | Gel | Time (min) | (Initial) | (Final) | Δ Shade |
| HE22 | Example I | 30 | B3 | A2 | 2 |
| HE23 | Example I | 30 | A3 | A2 | 4 |
| HE24 | Example I | 30 | B3 | D4 | 3 |
| HE25 | Example I | 30 | D3 | B2 | 7 |
| HE26 | Example I | 30 | B3 | A2 | 6 |
| HE108 | Example I | 3 × 20 min | A3.5 | A3 | 3 |
| HE109 | Example I | 3 × 20 min | A4 | D3 | 5 |
| HE110 | Example I | 3 × 20 min | A3.5 | A3.5 | 0 |
| HE111 | Example I | 3 × 20 min | A4 | A3 | 6 |
| HE112 | Example I | 3 × 20 min | A4 | A3.5 | 3 |

### Example 4

A second tooth whitening composition is prepared according to the following formula:

| | Percent |
|---|---|
| Ingredient | by Weight |
| Water | 61.300 |
| Glycerin (Dow) | 5.000 |
| 1-hydroxyethylidine-1,1-diphosphonic acid | 0.500 |
| (Dequest 2010, Solutia) | |
| Potassium Stannate Trihydrate (Goldschmidt) | 0.100 |
| Sodium Saccharin | 0.600 |
| Hydrogen peroxide (35% w/w solution from Solvay) | 15.000 |
| Carbopol 974P (Noveon) | 5.000 |
| PVP (Kollidon 25, BASF) | 5.000 |
| PEG-60 Hydrogenated Castor Oil (Cremaphor RH60, BASF) | 4.000 |
| Flavor | 1.000 |
| Ammonium Hydroxide (29% Solution) | 2.500 |
| Total | 100.000 |

### Manufacturing Method:

Combine water and glycerin, add Dequest 2010, potassium stannate trihydrate and sodium saccharin; mix until completely dissolved. Add hydrogen peroxide solution and mix well. Add Carbopol all at once, mix with high agitation to disperse and dissolve. Transfer to planetary mixer and continue mixing until smooth. Adjust pH to 5.2-5.5 with ammonium hydroxide, added drop-wise over a period of at least 10 minutes. Add PVP all at once, mix until smooth (mix will lose much of the viscosity developed after Carbopol neutralization). Heat Cremophor RH-60 to melt, add flavor and mix. Add Cremophor/flavor blend, mix thoroughly and desecrate. Transfer to bulk containers or fill into syringes, brush or felt tip pens, or other suitable delivery device.

### Example 5

A clinical trial was conducted with 44 subjects to study the efficacy and safety of the formulation provided in Example 4, a 5.25% hydrogen peroxide gel, supplied in a brush-on pen for vital tooth bleaching. The objective of the study was to test the efficacy of the whitening pen as well as patient compliance due to its ease of use. The secondary objective was to evaluate any sensitivity of teeth or possible effect on the tissues of the oral cavity.

### Method:

The investigation divided the subjects into two groups, A and B, each including 22 subjects. Subjects in groups A & B completed a medical and dental history as well as informed consent forms. This was followed by an interview, oral examination, and an evaluation of the teeth shade using the Vita Pan Shade Guide. A random population was chosen with the majority having an average pre-whitening shade of A-3 (tab 9) as measured on the Vita Pan Shade Guide. Pregnant or nursing women and those subjects with severe or moderate periodontal disease and any other medical or dental complications were excluded. If enrolled, the shade was recorded and photographed using the Polaroid SLR 5 camera. Special attention was paid to keep the lighting constant in the same operating room, as well as maintaining identical settings on the camera.

The patients in Group A were given the brush-on pen with instructions to apply the gel twice daily every day except Sundays for two weeks. Group A patients were instructed to apply a thin film on the tooth surface. The patients were asked not to eat or drink anything for at least 15 minutes post application. Group A was instructed to contact the Center with any sensitivity issues or any other compliance questions.

Group B patients were instructed to visit the La Jolla BriteSmile Center twice daily for two weeks except Sundays since the Center was closed. Those in Group B had the gel placed on by a clinical investigator at the La Jolla BriteSmile Center and given the same instructions to not eat or drink anything for at least 15 minutes post application. Those in Group B were evaluated daily for sensitivity and any signs of oral irritations.

The shade changes for Groups A and B were evaluated at the conclusion of the study using the Vita Pan Shade in the following order of lightest to darkest: B1, A1, B2, D2, A2, C1, C2, D4, A3, D3, B3, A3.5, B4, C3, A4, C4. These shades were then assigned a numerical number of 1 through 16, B1 being number 1.

### Results:

This study demonstrated an average improvement of 5 shades as measured on the Vita Shade Guide. No sensitivity or any other complications were noted in any of the subjects.

The results of the efficacy were analyzed using the Vita Pan Shade Guide with numerical values of 1 through 16.

Table 2 shows the average shade change statistics for Groups A and B.

TABLE 2

| | Average Shade change | Average Shade change |
|---|---|---|
| Group | (total) | (A3 and darker) |
| A | 4.5 +/− 1.7 | 5.1 +/− 1.6 |
| B | 4.3 +/− 1.3 | 4.6 +/− 1.3 |
| P values (T- | 0.29 | 0.21 |
| test)* | | |

*P-values suggest that the populations are more likely to be similar than not and that patient compliance was high.

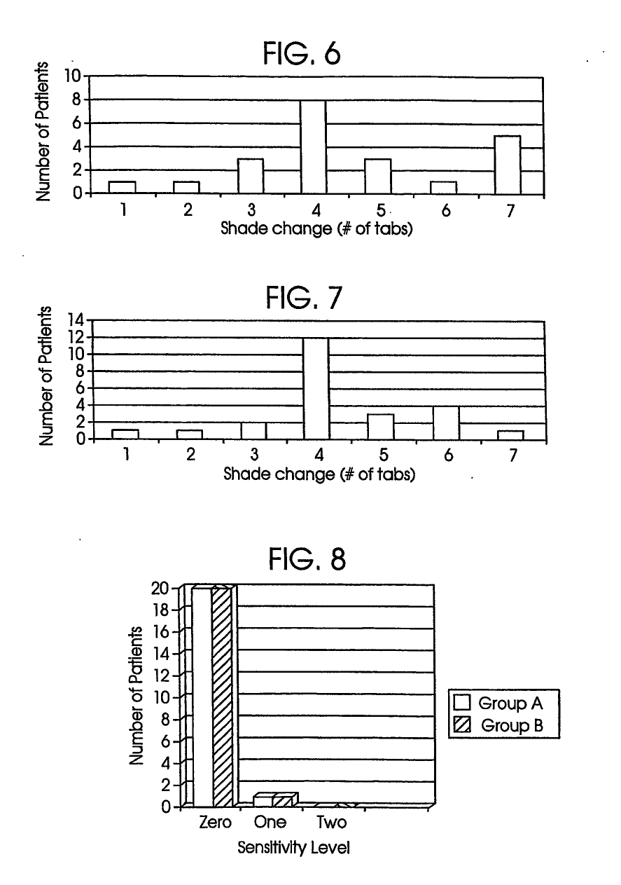

As shown in Table 2 and depicted in FIGS. 6 and 7, these results demonstrate that the formulation of Example 4 produced an average of 5 shades for patients A3 and darker and 4.5 shades for the total sample.

The safety was analyzed by evaluating the effect on the oral tissue and by measuring the sensitivity that was reported. The final oral exam evaluated the lips, the palate, the gingival mucosa and surrounding tissue and glands as well as a complete oral cancer screen. The sensitivity was evaluated by reporting none, mild, moderate or severe with each given a numerical value of 0 to 3 with severe being 3. (FIG. 8).

### Discussion:

The efficacy of the 5% hydrogen peroxide gel of Example 4 in the brush-on pen was measured for both Group A and Group B using the Vita Pan Shade Guide scores. The average shade improvement was not significantly different between both groups and averaged 5 shades for patients A3 and darker and 4.5 shades for the total sample. Both groups reported no sensitivity. Every participant in the study noticed an improvement subjectively and some indicated that they started noticing improvement within days of using the brush-on pen.

The results are significant in both the efficacy and the low percentage of sensitivity when compared to any other available tooth whitening product. Also given the similarity in shade improvement of both groups this maybe indicative of good patient compliance. On an exit interview patients rated the brush-on pen as an 8+ on a scale of 1-10, 10 being highest.

### Example 6

The effect of the presence of various salts on the viscosity of the therapeutic dental compositions in Table 3 was assessed.

TABLE 3

| | Percent (w/w) | | | |
|---|---|---|---|---|
| Ingredient | Form. 1 | Form. 2 | Form. 3 | Form. 4 |
| Water | 67.837 | 70.900 | 66.900 | 61.900 |
| Glycerine 99.7% | 5.000 | 5.000 | 5.000 | 5.000 |
| Etidronic acid | 0.300 | 0.500 | 0.500 | 0.500 |
| Sodium acid pyrophosphate | 0.100 | | | |
| Potassium Stannate Trihydrate | 0.020 | 0.100 | 0.100 | 0.100 |
| H2O2 (35% solution) | 17.143 | 15.000 | 15.000 | 15.000 |
| Carbopol 974P-NF | 5.000 | 5.000 | 5.000 | 5.000 |
| PVP K-25 | | 1.000 | 5.000 | 10.000 |
| Ammonium Hydroxide 29% | 4.600 | 2.500 | 2.500 | 2.500 |
| Total | 100.000 | 100.000 | 100.000 | 100.000 |

The measurements were made with a Brookfield Cone-Plate Viscometer at approximately 25 degrees Celsius. The results are depicted in Table 4. As depicted in Table 4, the viscosity of Formulations 3 and 4, which included 5.0% Carbopol/5.0% PVP and 5.0% Carbopol/10.0% PVP, respectively, significantly decreased in the presence of an alkali metal ion. The alkali metal ion did not have a significant effect on viscosity, however, in Formulations 1 and 2, which included 5.0% Carbopol in combination with 1.0% or no PVP.

TABLE 4

| | Percent | Percent | Percent | | | |
|---|---|---|---|---|---|---|
| Formu- | Carbopol | PVP | Na | Percent | Percent | Viscosity |
| lation | 974P | K-25 | Saccharin | NaCl | KCl | (cps) |
| 1 | 5.00% | 0.00% | | | | 130,000 |
| | 5.00% | 0.00% | 0.60% | | | 115,000 |
| | 5.00% | 0.00% | | 0.60% | | 113,000 |
| | 5.00% | 0.00% | | | 0.60% | 110,000 |
| 2 | 5.00% | 1.00% | | | | 128,000 |
| | 5.00% | 1.00% | 0.60% | | | 123,000 |
| | 5.00% | 1.00% | | 0.60% | | 126,000 |
| | 5.00% | 1.00% | | | 0.60% | 131,000 |
| 3 | 5.00% | 5.00% | | | | 143,000 |
| | 5.00% | 5.00% | 0.60% | | | 67,521 |
| | 5.00% | 5.00% | | 0.60% | | 72,078 |
| | 5.00% | 5.00% | | | 0.60% | 53,438 |
| 4 | 5.00% | 10.00% | | | | 145,000 |
| | 5.00% | 10.00% | 0.60% | | | 43,081 |
| | 5.00% | 10.00% | | 0.60% | | 50,952 |
| | 5.00% | 10.00% | | | 0.60% | 47,224 |

### Example 7

A second tooth whitening composition is prepared according to the guidelines in Table 5:

TABLE 5

| | | | Percent |
|---|---|---|---|
| Ingredient | Examples | Function in Product | (w/w) |
| Water | Water | Diluent/Carrier Fluid | 10-98.7% |
| Moisture Sensitive | carboxypolymethylene/ | Thickens product in the presence | 0.01%-50% |
| Polymer Complex | PVP | of additional moisture | |
| Optional pH/Ion | carboxypolymethylene | Thickens in response to increase | 0.01-10% |
| Sensitive Polymer | PVP/maleic acid anhydride | in pH | |
| | copolymer | | |
| | Polycarboxylates | | |
| | Gellan gum | | |
| | Cellulose acetate phthalate | | |
| Optional Temperature | Methylcellulose | Gels in response to increase in | 0.01%-10% |
| Sensitive Polymer | Hydroxypropyl | temperature above about 30° C. | |
| | methylcellulose | | |
| | Poly(oxyethylene)- | | |
| | poly(oxypropylene) block | | |
| | copolymer | | |
| Optional Polyol | Glycerin | Water retention/gel texture | 1-50% |
| | Propylene Glycol | modification | |
| | Polyethylene Glycol | | |
| | Sorbitol | | |
| | Mannitol | | |
| Therapeutic Agent | Sodium fluoride | Anticaries | 0.01-20% |
| | Hydrogen peroxide | Tooth whitening/antibacterial | |
| | Chlorhexidine digluconate | Antiplaque/antigingivitis | |
| | Sodium tripolyphosphate | Tartar control | |
| | Potassium nitrate | Tooth desensitizser | |

### Example 8

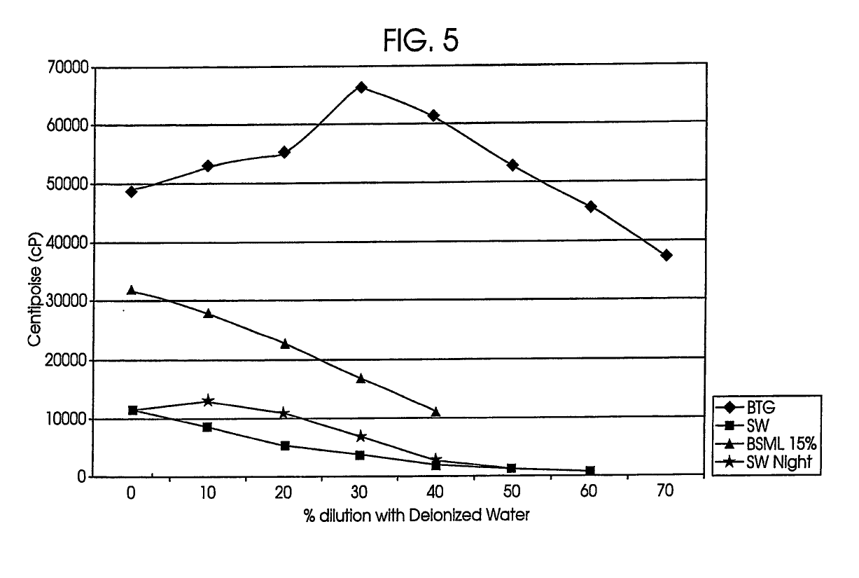

The dilution viscosity of the therapeutic dental composition of Example 7 was compared to several different gels. The measurements were made with a Brookfield Cone-Plate Viscometer at approximately 25 degrees Celsius. The results are depicted in FIG. 5. In FIG. 5, “BTG” represents the inventive composition of Example 7, while SW and SW Night (Simply White and Simply White Night) are Colgate's commercial brush-on products, and the BSML 15% is the current BriteSmile 15% Procedure Gel. As depicted in FIG. 5, the viscosity of BTG increases to a peak of approximately 65,000 cP as the composition is diluted to up to approximately 30%, whereas the viscosities of the prior art compositions decrease as dilution increases.

### Example 9

Composition of a one component tooth whitening formulation suitable for use as the second tooth whitening composition of the present invention.

The formulations below utilized ultrapure components to avoid destabilization caused by metal ion contaminants. The chelating agent used here is one of disodium EDTA (9C), citric acid (9B), and sodium acid pyrophosphate (9F). The pH is modified using one of sodium hydroxide monohydrate (9A, 9B, 9C), ammonium hydroxide (9F, 9G), Tris(hydroxymethyl)aminomethane (9D), and triethanolamine (9E). Carbopol is a high molecular weight cross-linked polyacrylic acid thickening agent. Hydrogen peroxide is used as the oxidizing agent.

TABLE 6

| | Example 9 | | | | | | |
|---|---|---|---|---|---|---|---|
| | A | B | C | D | E | F | G |
| Ingredient | WEIGHT PERCENT | | | | | | |
| Distilled Water | 86.41 | 86.21 | 86.31 | 72.80 | 79.52 | 86.50 | 73.81 |
| 1-Hydroxyethylidene-1,1- | 0.02 | 0.02 | 0.02 | 0.03 | 0.02 | 0.30 | 0.40 |
| diphosphonic acid | | | | | | | |
| Sodium stannate trihydrate | 0.02 | 0.02 | 0.02 | 0.03 | 0.02 | 0.05 | 0.05 |
| Citric acid | — | 0.20 | — | — | — | — | 0.10 |
| Calcium disodium EDTA | — | — | 0.10 | — | — | — | — |
| Sodium acid pyrophosphate | — | — | — | — | — | 0.30 | — |
| Hydrogen Peroxide 35% | 10.30 | 10.30 | 10.30 | 17.14 | 17.14 | 8.60 | 17.14 |
| Carbopol 974P (BF Goodrich) | 2.50 | 2.50 | 2.50 | 5.00 | — | 3.00 | 5.00 |
| Carbopol 934P (BF Goodrich) | — | — | — | — | 2.00 | — | — |
| Sodium Hydroxide | to pH 7.0 | to pH 7.0 | to pH 7.0 | — | — | — | — |
| Monohydrate | | | | | | | |
| Ammonium hydroxide 28% | — | — | — | — | — | to pH 6.5 | to pH 8.5 |
| Tris(hydroxymethyl) | — | — | — | to pH 8.0 | — | — | — |
| aminomethane | | | | | | | |
| Triethanolamine | — | — | — | — | to pH 6.0 | — | — |
| Total | 100 | 100 | 100 | 100 | 100 | 100 | 100 |
| pH @ 25 deg. C. | 7.0 | 7.0 | 7.0 | 8.0 | 6.0 | 6.5 | 8.5 |

The above formulations were prepared by dissolving 1-hydroxyethylidene-1,1-diphosphonic acid and sodium stannate trihydrate in distilled water using a Kynar-coated propeller-type agitator (reserving enough water, if necessary, to dissolve the neutralizer in the final step). Hydrogen peroxide was then added slowly to this mixture. Carbopol 974P was then added to the distilled water/stabilizer/hydrogen peroxide mixture slowly while a vortex was formed with the agitator blade. This dispersion was then placed in a Kynar-coated vacuum double planetary mixer to which the pH adjusting agent was added slowly to affect neutralization of the Carbopol 974P and to adjust the final composition pH. The resulting composition was a transparent, viscous gel and was packaged in foil/plastic laminate tubes having a polyethylene product contact liner.

### Example 10

In vivo demonstration of tooth bleaching

Six volunteers aged 25 to 43 were separated into two groups of two and custom dental trays were fashioned for each participant in the study.

One group was given an unmarked 2 oz. tube containing the composition of Example 9B and instructed to place a small amount of tooth-bleaching material into the tray, position the tray over the teeth, and leave the tray in place for 20 minutes. Patients were instructed to repeat this procedure twice a day for one week, for a total of 14 treatments and 280 minutes total tooth whitener exposure time.

The second group was given an unmarked 2 oz. tube of Opalescence 10% Carbamide Peroxide tooth-bleaching gel and instructed as above, with the exception of the duration of the bleaching procedure to be 60 minutes. Patients were instructed to repeat the procedure twice a day for one week, for a total of 14 treatments and 840 minutes total tooth-bleaching exposure time.

The results of direct tooth surface (upper left central incisor) color measurements, both before and after treatments are recorded in Table 7 below.

TABLE 7

| | | Treatment | | | | | | | |
|---|---|---|---|---|---|---|---|---|---|
| Patient | | Time | Initial Color | | | Final Color | | | |
| # | Product/Example | (min.) | L | a | b | L | a | b | ΔE |
| 1 | 7B | 280 | 53.76 | 4.65 | 11.65 | 60.34 | 0.97 | 8.80 | 8.06 |
| 2 | 7B | 280 | 49.42 | 2.97 | 9.48 | 56.99 | 0.46 | 7.38 | 8.25 |
| 3 | 7B | 280 | 51.26 | 2.33 | 8.25 | 55.63 | 0.87 | 4.99 | 5.65 |
| 4 | Opalescence | 840 | 52.78 | 1.75 | 6.14 | 57.26 | 1.42 | 2.10 | 6.04 |
| 5 | Opalescence | 840 | 56.35 | 1.79 | 5.21 | 59.13 | 0.65 | 2.44 | 4.09 |
| 6 | Opalescence | 840 | 55.71 | 2.72 | 7.10 | 58.60 | 1.09 | 4.75 | 4.07 |

The average ΔE for the Example 9B group was 7.32, whereas the average ΔE for the Opalescence group was 4.73.

### Example 11

The tooth surface of a subject will be effectively whitened and the whiteness maintained for a longer period than the prior art methods by following the below protocol.

### Materials and Methods:

Approximately 50 subjects will be chosen to participate in this study with approximately 40 subjects who are at least A3 or darker. The first tooth whitening composition will include the formulation of Example 2. The second tooth whitening composition will include the formulation of Example 4 and will be administered in a pen device.

Application of the first tooth whitening composition will be performed by dental personnel in a dental office. Shade measurements will be taken prior to commencement of this study and evaluated on the VITA® shade guide. The subject will first brush the teeth with a pre-whitening toothpaste. The dental personnel will then place a cheek retractor and bite block in the subject's mouth. Next, the dental personnel will apply barrier material to the subject's oral cavity to protect the gingival margins from the oxidizing agent (hydrogen peroxide) in the first tooth whitening composition. The first tooth whitening composition will be applied by the dental personnel onto the subject's tooth surface and will remain on the teeth for about 30 minutes. After the 30 minutes, the dental personnel will remove the first tooth whitening composition from the tooth surface and the barrier material, cheek retractor and bite block from the oral cavity. Shade measurements will be taken again immediately after. Lastly, the dental personnel will provide the subject with the second tooth whitening composition and instruct the subject to use the second tooth whitening composition twice daily at home for 14 days. The subject will return to the dental office for further shade measurements one week and two weeks after the initial treatment with the first tooth whitening composition

A number of embodiments of the invention have been described. Nevertheless, it will be understood that various modifications may be made without departing from the spirit and scope of the invention. Accordingly, other embodiments are within the scope of the following claims, and as various changes can be made to the above compositions, formulations, combinations, and methods without departing from the scope of the invention, it is intended that all matter contained in the above description be interpreted as illustrative and not in a limiting sense. All patent documents and references listed herein are incorporated by reference.

I claim:

1. A method of whitening teeth in a subject, comprising:
applying a first tooth whitening composition to the teeth of the subject for a predetermined period of time; and
applying a second tooth whitening composition to the teeth of the subject in predetermined intervals;
wherein at least one of the first and second tooth whitening compositions is dispensed from a dental delivery device.

2. The method of claim 1, wherein the first tooth whitening composition comprises a first oxidizing agent.

3. The method of claim 2, wherein the first oxidizing agent is selected from the group consisting of hydrogen peroxide, carbamide peroxide, alkali metal peroxides, chlorine dioxide, sodium chlorite, alkali metal percarbonates, and alkali metal perborates.

4. The method of claim 2, wherein the first oxidizing agent comprises hydrogen peroxide in an amount from about 10.0% to about 36.0% weight to weight of the first tooth whitening composition.

5. The method of claim 2, wherein the first oxidizing agent comprises hydrogen peroxide in an amount of about 15.0% weight to weight of the first tooth whitening composition.

6. The method of claim 1, wherein the first tooth whitening composition further comprises a first stabilizer.

7. The method of claim 6, wherein the first stabilizer is selected from the group consisting of 1-hydroxyethylidene-1,1-diphosphonic acid, sodium stannate trihydrate, sodium acid pyrophosphate, ethylenediamine tetraacetic acid (EDTA), diethylenetriamine pentaacetic acid (DETPA), nitrilotriacetic acid (NTA), ethylenediamine tetra(methylenephosphonic acid), diethylenetriamine penta(methylenephosphonic acid), sorbitol, xylitol, mannitol, maltitol, lactitol, alkali metal pyrophosphates and alkali metal polyphosphates.

8. The method of claim 1, wherein the predetermined period of time ranges from about five minutes to about one hour.

9. The method of claim 1, wherein the second tooth whitening composition comprises a second oxidizing agent.

10. The method of claim 9, wherein the second oxidizing agent is selected from the group consisting of hydrogen peroxide, carbamide peroxide, alkali metal peroxides, chlorine dioxide, sodium chlorite, alkali metal percarbonates, and alkali metal perborates.

11. The method of claim 9, wherein the second oxidizing agent comprises hydrogen peroxide in an amount from about 1.0% to about 10.0% weight to weight of the second tooth whitening composition.

12. The method of claim 9, wherein the second oxidizing agent comprises hydrogen peroxide in an amount of about 5.0% weight to weight of the second tooth whitening composition.

13. The method of claim 1, wherein the second tooth whitening composition further comprises a second stabilizer.

14. The method of claim 13, wherein the second stabilizer is selected from the group consisting of 1-hydroxyethylidene-1,1-diphosphonic acid, sodium stannate trihydrate, sodium acid pyrophosphate, ethylenediamine tetraacetic acid (EDTA), diethylenetriamine pentaacetic acid (DETPA), nitrilotriacetic acid (NTA), ethylenediamine tetra(methylenephosphonic acid), diethylenetriamine penta(methylenephosphonic acid), sorbitol, xylitol, mannitol, maltitol, lactitol, alkali metal pyrophosphates and alkali metal polyphosphates.

15. The method of claim 1, wherein the predetermined interval ranges from about one to about six times per day.

16. A method of whitening teeth in a subject, comprising:
applying a first tooth whitening composition to the teeth of the subject for a predetermined period of time; and
providing a second tooth whitening composition to the subject and instructing the subject to apply the second tooth whitening composition in predetermined intervals of time;
wherein at least one of the first and second tooth whitening compositions is dispensed from a dental delivery device.

17. The method of claim 16, wherein the first tooth whitening composition comprises a first oxidizing agent.

18. The method of claim 17, wherein the first oxidizing agent is selected from the group consisting of hydrogen peroxide, carbamide peroxide, alkali metal peroxides, chlorine dioxide, sodium chlorite, alkali metal percarbonates, and alkali metal perborates.

19. The method of claim 17, wherein the first oxidizing agent comprises hydrogen peroxide in an amount from about 10.0% to about 36.0% weight to weight of the first tooth whitening composition.

20. The method of claim 17, wherein the first oxidizing agent comprises hydrogen peroxide in an amount of about 15.0% weight to weight of the first tooth whitening composition.

21. The method of claim 16, wherein the first tooth whitening composition further comprises a first stabilizer.

22. The method of claim 21, wherein the first stabilizer is selected from the group consisting of 1-hydroxyethylidene-1,1-diphosphonic acid, sodium stannate trihydrate, potassium stannate trihydrate, sodium acid pyrophosphate, ethylenediamine tetraacetic acid (EDTA), diethylenetriamine pentaacetic acid (DETPA), nitrilotriacetic acid (NTA), ethylenediamine tetra(methylenephosphonic acid), diethylenetriamine penta(methylenephosphonic acid), sorbitol, xylitol, mannitol, maltitol, lactitol, alkali metal pyrophosphates and alkali metal polyphosphates.

23. The method of claim 16, wherein the predetermined period of time ranges from about five minutes to about one hour.

24. The method of claim 16, wherein the second tooth whitening composition comprises a second oxidizing agent.

25. The method of claim 24, wherein the second oxidizing agent is selected from the group consisting of hydrogen peroxide, carbamide peroxide, alkali metal peroxides, chlorine dioxide, sodium chlorite, alkali metal percarbonates, and alkali metal perborates.

26. The method of claim 24, wherein the second oxidizing agent comprises hydrogen peroxide in an amount from about 1.0% to about 10.0% weight to weight of the second tooth whitening composition.

27. The method of claim 24, wherein the second oxidizing agent comprises hydrogen peroxide in an amount of about 5.0% weight to weight of the second tooth whitening composition.

28. The method of claim 16, wherein the second tooth whitening composition further comprises a second stabilizer.

29. The method of claim 28, wherein the second stabilizer is selected from the group consisting of 1-hydroxyethylidene-1,1-diphosphonic acid, sodium stannate trihydrate, potassium stannate trihydrate, sodium acid pyrophosphate, ethylenediamine tetraacetic acid (EDTA), diethylenetriamine pentaacetic acid (DETPA), nitrilotriacetic acid (NTA), ethylenediamine tetra(methylenephosphonic acid), diethylenetriamine penta(methylenephosphonic acid), sorbitol, xylitol, mannitol, maltitol, lactitol, alkali metal pyrophosphates and alkali metal polyphosphates.

30. The method of claim 16, wherein the predetermined interval ranges from about one to about six times per day.

31. A kit for whitening teeth in a subject, comprising:
a first tooth whitening composition comprising from about 10.0% to about 36.0% hydrogen peroxide weight to weight of the first tooth whitening composition;
a second tooth whitening composition comprising from about 1.0% to about 10.0% hydrogen peroxide weight to weight of the second tooth whitening composition;
a dental delivery device configured to dispense the second tooth whitening composition; and
a set of instructions.

32. The kit of claim 31, wherein the hydrogen peroxide is present in an amount of about 15.0% weight to weight of the first tooth whitening composition.

33. The kit of claim 31, wherein the first tooth whitening composition further comprises a first stabilizer.

34. The kit of claim 33, wherein the first stabilizer is selected from the group consisting of 1-hydroxyethylidene-1,1-diphosphonic acid, sodium stannate trihydrate, potassium stannate trihydrate, sodium acid pyrophosphate, ethylenediamine tetraacetic acid (EDTA), diethylenetriamine pentaacetic acid (DETPA), nitrilotriacetic acid (NTA), ethylenediamine tetra(methylenephosphonic acid), diethylenetriamine penta(methylenephosphonic acid), sorbitol, xylitol, mannitol, maltitol, lactitol, alkali metal pyrophosphates and alkali metal polyphosphates.

35. The kit of claim 31, wherein the first tooth whitening composition further comprises a thickener.

36. The kit of claim 35, wherein the thickener is selected from the group consisting of carboxypolymethylene, polyacrylic acid polymers and copolymers, hydroxypropylcellulose, cellulose ethers, salts of poly(methyl vinyl ether-co-maleic anhydride), polyvinyl pyrrolidone, poly(vinylpyrrolidone-co-vinyl acetate), silicon dioxide, fumed silica, and stearic acid esters.

37. The kit of claim 31, wherein the first tooth whitening composition further comprises a first alkaline pH adjusting agent.

38. The kit of claim 37, wherein the first alkaline pH adjusting agent is selected from the group consisting of sodium hydroxide, potassium hydroxide, ammonium hydroxide, sodium carbonate, potassium carbonate, sodium phosphate di- and tri-basic, potassium phosphate di- and tri-basic, sodium tripolyphosphate, tris(hydroxymethyl)aminomethane, triethanolamine, and polyethylenimine.

39. The kit of claim 31, wherein the first tooth whitening composition further comprises a first secondary therapeutic agent selected from the group consisting of antimicrobial agents, anti-inflammatory agents, tooth desensitizers, anticaries agents, tartar control agents, tooth and gum surface protectants, tooth stain prevention agents and agents effective against dental plaque, halitosis, gingivitis, periodontal disease, oral ulcers and other diseases, afflictions or symptoms of the oral cavity.

40. The kit of claim 31, wherein the hydrogen peroxide is present in an amount of about 5.0% weight to weight of the second tooth whitening composition.

41. The kit of claim 31, wherein the second tooth whitening composition further comprises a second stabilizer.

42. The kit of claim 41, wherein the second stabilizer is selected from the group consisting of 1-hydroxyethylidene-1,1-diphosphonic acid, sodium stannate trihydrate, sodium acid pyrophosphate, ethylenediamine tetraacetic acid EDTA), diethylenetriamine pentaacetic acid (DETPA), nitrilotriacetic acid (NTA), ethylenediamine tetra(methylenephosphonic acid), diethylenetriamine penta(methylenephosphonic acid), sorbitol, xylitol, mannitol, maltitol, lactitol, alkali metal pyrophosphates and alkali metal polyphosphates.

43. The kit of claim 31, wherein the second tooth whitening composition further comprises a second alkaline pH adjusting agent.

44. The kit of claim 43, wherein the second alkaline pH adjusting agent is selected from the group consisting of sodium hydroxide, potassium hydroxide, ammonium hydroxide, sodium carbonate, potassium carbonate, sodium phosphate di- and tri-basic, potassium phosphate di- and tri-basic, sodium tripolyphosphate, tris(hydroxymethyl)aminomethane, triethanolamine, and polyethylenimine.

45. The kit of claim 31, wherein the second tooth whitening composition further comprises a second secondary therapeutic agent selected from the group consisting of antimicrobial agents, anti-inflammatory agents, tooth desensitizers, anticaries agents, tartar control agents, tooth and gum surface protectants, tooth stain prevention agents and agents effective against dental plaque, halitosis, gingivitis, periodontal disease, oral ulcers and other diseases, afflictions or symptoms of the oral cavity.

46. The kit of claim 31, wherein the second tooth whitening composition further comprises a moisture responsive gel carrier.

47. The kit of claim 46, wherein the moisture responsive gel carrier comprises from about 80.0% (w/w) to about 99.99% (w/w) of the second tooth whitening composition.

48. The kit of claim 46, wherein the moisture responsive gel carrier comprises a moisture sensitive polymer complex, water, and a water soluble salt.

49. The kit of claim 48, wherein the moisture sensitive polymer complex comprises carboxypolymethylene and polyvinylpyrrolidone.

50. The kit of claim 48, wherein the water soluble salt is selected from the group consisting of sodium saccharin, sodium chloride, potassium chloride, and ammonium chloride.

51. The kit of claim 48, wherein the moisture sensitive polymer complex is present in an amount of about 0.01% (w/w) to about 50.0% (w/w) of the composition.

52. The kit of claim 48, wherein the moisture sensitive polymer complex is present in an amount of about 0.01% (w/w) to about 10.0% (w/w) of the composition.

53. The kit of claim 31, wherein the dental delivery device comprises:
an applicator for applying the first tooth whitening composition to the tooth surface;
an actuator; and
a reservoir located between the applicator and the actuator and configured to store the first tooth whitening composition,
wherein the actuator is configured to dispense the first tooth whitening composition from the reservoir to the applicator.

54. The kit of claim 31, wherein the dental delivery device comprises:
an applicator for applying the second tooth whitening composition to the tooth surface;
an actuator; and
a reservoir located between the applicator and the actuator and configured to store the second tooth whitening composition,
wherein the actuator is configured to dispense the second tooth whitening composition from the reservoir to the applicator.

55. The kit of claim 53 or 54, wherein the applicator is selected from the group consisting of a brush, a felt tip, a roller ball, and a non-woven pad.

56. The kit of claim 53 or 54, wherein the actuator is selected from the group consisting of a click mechanism, a twist and ratchet mechanism, and a push button.

57. The kit of claim 53 or 54, wherein the applicator comprises a brush and the actuator comprises a push button, wherein the push button dispenses the composition to the brush applicator.

58. The kit of claim 53 or 54, wherein the applicator comprises a brush and the actuator comprises a twist mechanism, wherein the twist mechanism dispenses the composition to the brush applicator.

59. The kit of claim 53 or 54, wherein the applicator comprises a felt tip and the actuator comprises a push button, wherein the push button dispenses the composition to the felt tip applicator.

60. The kit of claim 53 or 54, wherein the applicator comprises a felt tip and the actuator comprises a twist mechanism, wherein the twist mechanism dispenses the composition to the felt tip applicator.

61. A two component tooth whitening system, comprising:
a first tooth whitening composition comprising
from about 10.0% to about 36.0% hydrogen peroxide weight to weight of the first tooth whitening composition; and
from about 64.0% to about 90.0% carrier weight to weight of the first tooth whitening composition; and
a second tooth whitening composition comprising
from about 1.0% to about 10.0% hydrogen peroxide weight to weight of the second tooth whitening composition; and
from about 90.0% to about 99.0% moisture responsive gel carrier weight to weight of the second tooth whitening composition.

62. A liquid oral therapeutic dental composition, that increases in viscosity upon contact with moisture following application to an oral cavity surface, comprising:
a moisture responsive gel carrier comprising a moisture sensitive polymer complex and a water-soluble salt; and
a therapeutic agent dispersed in the responsive gel carrier.

63. The composition of claim 62, wherein the moisture responsive gel carrier further comprises a thermally responsive polymer.

64. The composition of claim 62, wherein the moisture responsive gel carrier further comprises a pH or ion responsive polymer.

65. The composition of claim 62, wherein the therapeutic agent is selected from the group consisting of antimicrobial agents, tooth whiteners, anti-inflammatory agents, tooth desensitizers, anticaries agents, tartar control agents, tooth and gum surface protectants, tooth stain prevention agents and agents effective against dental plaque, halitosis, gingivitis, periodontal disease, oral ulcers and other diseases, afflictions or symptoms of the oral cavity.

66. The composition of claim 62, wherein the therapeutic agent comprises a tooth whitener.

67. The composition of claim 66, wherein the tooth whitener is selected from the group consisting of an alkali metal percarbonate, carbomide peroxide, sodium perborate, potassium persulfate, calcium peroxide, zinc peroxide, chlorine dioxide, sodium chlorite, a hydrogen peroxide complex, hydrogen peroxide and mixtures of any of the foregoing.

68. The composition of claim 66, wherein the tooth whitener comprises about 0.01% (w/w) to about 20.0% (w/w) of hydrogen peroxide.

69. The composition of claim 66, wherein the tooth whitener comprises about 2.0% (w/w) to about 30.0% (w/w) of carbamide peroxide.

70. The composition of claim 62, further comprising water present in an amount of about 10.0% (w/w) to about 98.7% (w/w) of the composition.

71. The composition of claim 62, wherein the moisture responsive gel carrier comprises from about 80.0% (w/w) to about 99.99% (w/w) of the composition.

72. The composition of claim 62, wherein the moisture sensitive polymer complex comprises carboxypolymethylene and polyvinylpyrrolidone.

73. The composition of claim 62, wherein the water soluble salt is selected from the group consisting of sodium saccharin, sodium chloride, potassium chloride, and ammonium chloride.

74. The composition of claim 63, wherein the temperature sensitive polymer comprises methylcellulose.

75. The composition of claim 63, wherein the temperature sensitive polymer comprises hydroxypropyl methylcellulose.

76. The composition of claim 63, wherein the temperature sensitive polymer comprises a poly(oxyethylene)-poly(oxypropylene) block copolymer.

77. The composition of claim 62, wherein the moisture sensitive polymer complex is present in an amount of about 0.01% (w/w) to about 50.0% (w/w) of the composition.

78. The composition of claim 62, wherein the moisture sensitive polymer is present in an amount of about 0.01% (w/w) to about 10.0% (w/w) of the composition.

79. A therapeutic dental delivery device for treating a condition in an oral cavity of a subject, comprising:
a liquid oral therapeutic dental composition;
an applicator for applying the composition to the oral cavity;
an actuator; and
a reservoir located between the applicator and the actuator and configured to store the composition,
wherein the actuator is configured to dispense the composition from the reservoir to the applicator.

80. The device of claim 79, wherein the therapeutic dental composition comprises a moisture responsive gel carrier and a therapeutic agent dispersed in the moisture responsive gel carrier.

81. The device of claim 80, wherein the moisture responsive gel carrier comprises a moisture sensitive polymer complex and a water soluble salt.

82. The device of claim 79, wherein the applicator is selected from the group consisting of a brush, a felt tip, a roller ball, and a non-woven pad.

83. The device of claim 79, wherein the actuator is selected from the group consisting of a click mechanism, a twist and ratchet mechanism, and a push button.

84. The device of claim 79, wherein the applicator comprises a brush and the actuator comprises a push button, wherein the push button dispenses the composition to the brush applicator.

85. The device of claim 79, wherein the applicator comprises a brush and the actuator comprises a twist mechanism, wherein the twist mechanism dispenses the composition to the brush applicator.

86. The device of claim 79, wherein the applicator comprises a felt tip and the actuator comprises a push button, wherein the push button dispenses the composition to the felt tip applicator.

87. The device of claim 79, wherein the applicator comprises a felt tip and the actuator comprises a twist mechanism, wherein the twist mechanism dispenses the composition to the felt tip applicator.

88. The device of claim 81, wherein the moisture sensitive polymer complex comprises carboxypolymethylene and polyvinylpyrrolidone.

89. The device of claim 80, wherein the therapeutic agent comprises a peroxide.

90. The device of claim 80, wherein the therapeutic agent comprises hydrogen peroxide.

91. The device of claim 80, wherein the therapeutic agent comprises carbamide peroxide.

92. The device of claim 79, wherein the composition is present in an amount of from about 0.01 ml to 1.0 ml.

93. A therapeutic dental pen for treating a condition in an oral cavity of a subject, comprising:
a liquid oral therapeutic dental composition comprising a moisture responsive gel carrier and a therapeutic agent dispersed in the responsive gel carrier, wherein the gel carrier comprises a polymer complex including carboxypolymethylene and polyvinylpyrrolidone and a water soluble salt;
a brush-on applicator for applying the composition to the oral cavity;
a push button; and
a reservoir located between the applicator and the push button and configured to store the composition,
wherein the push button is configured to dispense the composition from the reservoir to the applicator.

94. The pen of claim 93, wherein the therapeutic agent comprises a peroxide.

95. The pen of claim 93, wherein the therapeutic agent comprises hydrogen peroxide.

96. The pen of claim 93, wherein the therapeutic agent comprises carbamide peroxide.

97. A dental therapeutic kit comprising:
a liquid oral therapeutic dental composition comprising a moisture responsive gel carrier and a therapeutic agent;
a delivery device comprising:
an applicator for applying the composition to the oral cavity;
an actuator; and
a reservoir located between the applicator and the actuator and configured to store the composition, wherein the actuator is configured to dispense the composition from the reservoir to the applicator; and
a set of instructions.

98. A method for whitening teeth of a subject in need thereof, comprising:
providing a therapeutic dental composition comprising a moisture responsive gel carrier and a therapeutic agent dispersed in the responsive gel carrier, wherein the gel carrier comprises a polymer complex including carboxypolymethylene and polyvinylpyrrolidone and a water soluble salt; and
applying the composition to a tooth of the subject.

99. A method of whitening teeth in a subject, comprising:
applying a tooth whitening composition to the teeth of the subject with a device comprising:
an applicator for applying the tooth whitening composition to the oral cavity;
an actuator; and
a reservoir located between the applicator and the actuator and configured to store the composition,
wherein the actuator is configured to dispense the composition from the reservoir to the applicator through a transport channel.

100. The method of claim 99, wherein the device comprises plastic.

101. The method of claim 99, wherein the components of the device that are in contact with the tooth whitening composition comprise a plastic coating.
